(19) United States
(12) Patent Application Publication
(10) Pub. No.: US 2016/0058399 A1
(43) Pub. Date: Mar. 3, 2016

(54) X RAY DIAGNOSTIC APPARATUS AND PUNCTURE NEEDLE INSERTION ASSISTANT METHOD

(71) Applicants: Kabushiki Kaisha Toshiba, Minato-ku (JP); Toshiba Medical Systems Corporation, Otawara-shi (JP)

(72) Inventors: Yusuke NARABU, Nasushiobara (JP); Toshiya WAKU, Yaita (JP); Ko FUCHIGAMI, Otawara (JP)

(73) Assignees: Kabushiki Kaisha Toshiba, Minato-ku (JP); Toshiba Medical Systems Corporation, Otawara-shi (JP)

(21) Appl. No.: 14/936,241

(22) Filed: Nov. 9, 2015

Related U.S. Application Data
(63) Continuation of application No. PCT/JP2014/062499, filed on May 9, 2014.

(30) Foreign Application Priority Data
May 9, 2013 (JP) 2013-099199

(51) Int. Cl. A61B 6/12; A61B 6/00

(52) U.S. Cl. CPC A61B 6/12; A61B 6/4441; A61B 6/461; A61B 6/467; A61B 6/54; A61B 6/5247; A61B 2019/5238

(57) ABSTRACT

An X-ray diagnostic apparatus according to an embodiment controls the imaging system in order to generate an X-ray image in which an imaging center line connecting a focus of an X-ray tube and a center position of an X-ray detection surface has a tilt angle of smaller than 90° with respect to a guideline connecting an input insertion target position and an input arrival target position; extracts from the image, a region of the needle inserted in an object; specifies an apparent length of the needle based on the region of the needle extracted from the X-ray image having the tilt angle of smaller than 90°; calculates an estimated insertion length of the needle inserted in the object based on the apparent length of the needle and the tilt angle; and notifies a user of information for assisting an insertion operation of the needle based on the estimated length.

This application is a Continuation application of PCT Application No. PCT/JP2014/062499, filed May 9, 2014 and based upon and claims the benefit of priority from the Japanese Patent Application No. 2013-099199, filed May 9, 2013, the entire contents of all of which are incorporated herein by reference.

### FIELD

Embodiments described herein relate generally to an X-ray diagnostic apparatus and a puncture needle insertion assistant method.

### BACKGROUND

It is common practice to insert a puncture needle into an object for medication of curative medicine to a target region such as a focus of disease, and treatment and examination of a target region.

In treatment or examination of this type, an image including a target region is sometimes fluoroscoped or imaged by an X-ray diagnostic apparatus, and an obtained real-time image is displayed on a display. While confirming the tip position of the puncture needle on the real-time image, a doctor performs a puncture needle insertion operation so that the puncture needle reaches the target region.

An object of embodiments is to assist insertion of a puncture needle into an object.

### BRIEF DESCRIPTION OF THE DRAWINGS

FIG. 1 is a block diagram of an X-ray diagnostic apparatus common to embodiments.

FIG. 2 is a schematic view for explaining rotation control of a C-arm by an imaging control circuit.

FIG. 3 is a view for explaining problems at the time of inserting a puncture needle.

FIG. 4 is a block diagram of an X-ray diagnostic apparatus according to the first embodiment.

FIG. 5A is a schematic view showing the positional relationship between an imaging circuit and an object in a parallel mode.

FIG. 5B is a schematic view showing the positional relationship between the imaging circuit and the object in a tilt mode.

FIG. 5C is a schematic view showing the positional relationship between the imaging circuit and the object in a lateral mode.

FIG. 6 is a block diagram showing the functional blocks of an assistant image generation circuit according to the first embodiment.

FIG. 7 is a view showing an example of a fluoroscopic image corresponding to the parallel mode according to the first embodiment.

FIG. 8 is a view showing an example of a fluoroscopic image corresponding to the tilt mode according to the first embodiment.

FIG. 9 is a flowchart showing the operation of the X-ray diagnostic apparatus upon switching from the parallel mode to the tilt mode.

FIG. 10 is an explanatory view for explaining a method of calculating an estimated insertion length.

FIG. 11 is a view showing an example of an assistant image generated by the assistant image generation circuit according to the first embodiment.

FIG. 12 is a block diagram showing an X-ray diagnostic apparatus according to the second embodiment.

FIG. 13 is a view showing an example of a fluoroscopic image corresponding to the parallel mode according to the second embodiment.

FIG. 14 is a view showing an example of a fluoroscopic image corresponding to the tilt mode according to the second embodiment.

FIG. 15 is an explanatory view for explaining a method of deciding a rotation direction by a rotation direction decision circuit.

FIG. 16 is an explanatory view for explaining a method of deciding the tilt direction of the C-arm by the rotation direction decision circuit.

FIG. 17 is a block diagram showing the functional blocks of an assistant image generation circuit according to the second embodiment.

FIG. 18 is a flowchart showing an example of the operation of the X-ray diagnostic apparatus according to the second embodiment.

FIG. 19 is a view showing an example of an assistant image generated by the assistant image generation circuit according to the second embodiment.

FIG. 20A is a flowchart showing part of the workflow of the first example using the X-ray diagnostic apparatus according to the second embodiment.

FIG. 20B is a flowchart showing a remaining part of the workflow of the first example using the X-ray diagnostic apparatus according to the second embodiment.

FIG. 21A is a flowchart showing part of the workflow of the second example using the X-ray diagnostic apparatus according to the second embodiment.

FIG. 21B is a flowchart showing a remaining part of the workflow of the second example using the X-ray diagnostic apparatus according to the second embodiment.

### DETAILED DESCRIPTION

An X-ray diagnostic apparatus according to an embodiment comprises an imaging system, a display, an imaging control circuit, a puncture needle extraction circuit, an insertion length calculation circuit and a notification circuit. The imaging system rotatably holds, by an arm, an X-ray tube which generates an X-ray, and an X-ray detector which detects an X-ray having passed through an object placed on a top, and generates data of an X-ray image. The display displays the X-ray image generated by the imaging system. The input circuit inputs an insertion target position and arrival target position of a puncture needle which is inserted into the object. The imaging control circuit controls the imaging system in order to generate an X-ray image in which an imaging center line connecting a focus of the X-ray tube and a center position of an X-ray detection surface of the X-ray detector has a tilt angle of smaller than 90° with respect to a guideline connecting the insertion target position and the arrival target position. The puncture needle extraction circuit extracts, from the X-ray image generated by the imaging system, a region of the puncture needle inserted in the object. The insertion length calculation circuit specifies an apparent length of the puncture needle based on the region of the puncture needle extracted from the X-ray image having the tilt angle of smaller than 90°, and calculate an estimated insertion length of the puncture needle inserted in the object based on the apparent length of the puncture needle and the tilt angle. The notification circuit notifies a user of assistant information for assisting an insertion operation of the puncture needle based on the estimated insertion length.

Several embodiments will be described with reference to the accompanying drawings.

In the following description, the same reference numerals denote the same constituent elements, and a repetitive description will be omitted.

First, an arrangement common to embodiments will be explained.

[Overall Arrangement of X-Ray Diagnostic Apparatus]

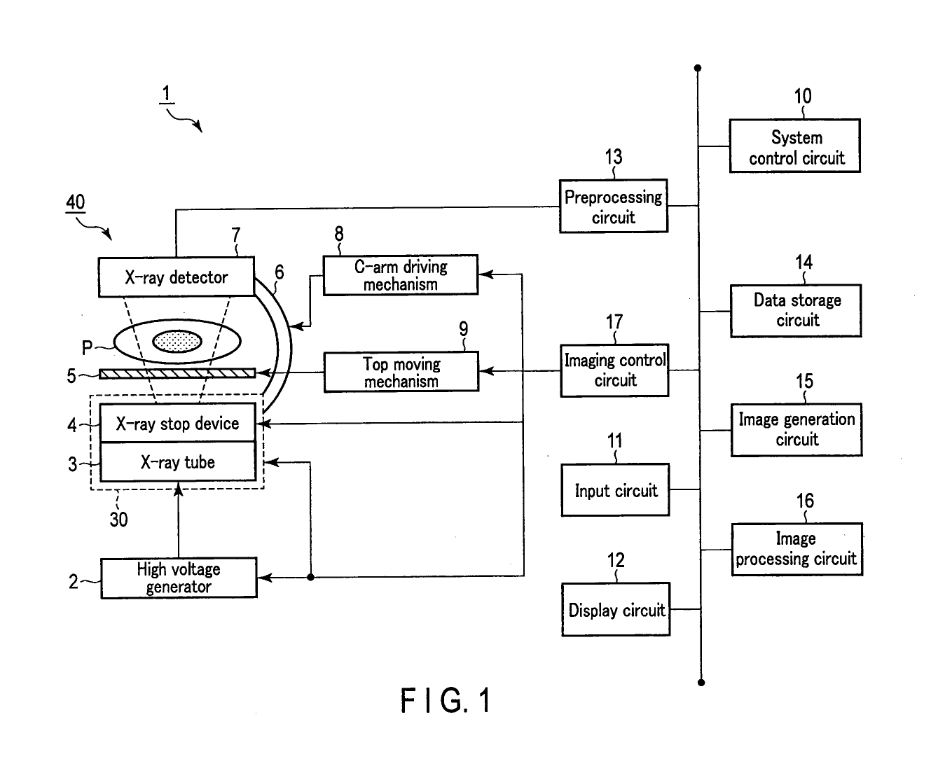

FIG. 1 is a block diagram of an X-ray diagnostic apparatus 1 common to embodiments.

As shown in FIG. 1, the X-ray diagnostic apparatus 1 includes a high voltage generator 2, an X-ray tube 3, an X-ray stop device 4, a top 5, a C-arm 6, an X-ray detector 7, a C-arm driving mechanism 8, a top moving mechanism 9, a system control circuit 10, an input circuit 11, a display circuit 12, a preprocessing circuit 13, a data storage circuit 14, an image generation circuit 15, an image processing circuit 16, and an imaging control circuit 17. The high voltage generator 2, the X-ray tube 3, the X-ray stop device 4, the X-ray detector 7, the C-arm driving mechanism 8, and the like constitute an imaging circuit 40. The X-ray tube 3 and the X-ray stop device 4 constitute an X-ray source device 30.

The high voltage generator 2 generates a tube voltage and tube current to be supplied to the X-ray tube 3. The X-ray tube 3 generates X-rays in response to supply of the tube current and application of the tube voltage from the high voltage generator 2. The high voltage generator 2 supplies, to the X-ray tube 3, a tube current and tube voltage complying with X-ray imaging conditions and X-ray fluoroscopy conditions set by a user or the like. X-ray fluoroscopy is an imaging method of continuously irradiating an object with X-rays smaller in dose than X-ray imaging (one-shot imaging). While viewing a moving image concerning an object that is provided by X-ray fluoroscopy, the user such as a doctor performs, e.g., a puncture operation and intervention. The X-ray stop device 4 is a device for limiting an X-ray irradiation range on the X-ray detection surface of the X-ray detector 7 to a region of interest of an object P to reduce unnecessary exposure of the object P. For example, the X-ray stop device 4 includes four slidable aperture blades, and limits the X-ray irradiation range by sliding these aperture blades.

The object P is placed on the top 5. The X-ray detector 7 includes a plurality of X-ray detection elements which detect X-rays having passed through the object P. Each of the X-ray detection elements converts a charge signal (analog signal) corresponding to incident X-rays into an image signal (digital signal), and outputs the digital signal to the preprocessing circuit 13. The C-arm 6 holds the X-ray tube 3, the X-ray stop device 4, and the X-ray detector 7 so that they face each other via the object P. An arm of another type such as an Ω-arm can also be used instead of the C-arm 6. The X-ray source device 30 and the X-ray detector 7 may be held by two holding circuits which independently hold the X-ray source device 30 and the X-ray detector 7, respectively.

The C-arm driving mechanism 8 is a device for rotating and moving the C-arm 6. The top moving mechanism 9 moves the top 5 in a horizontal direction parallel to the mount surface and a vertical direction perpendicular to the mount surface.

The preprocessing circuit 13 executes preprocessing on an image signal output from the X-ray detector 7. Preprocessing includes, e.g., sensitivity correction and dark current correction. Note that an image signal after preprocessing is also called projection data. The projection data is output to the data storage circuit 14 together with data of a projection angle.

The image generation circuit 15 generates data of an X-ray image based on an image signal having undergone preprocessing. The X-ray diagnostic apparatus 1 according to each embodiment (to be described later) mainly performs X-ray fluoroscopy. Thus, an X-ray image generated by the image generation circuit 15 will be called a fluoroscopic image. Data of the fluoroscopic image is output to the data storage circuit 14 and the display circuit 12.

The data storage circuit 14 stores data of a fluoroscopic image generated by the image generation circuit 15. The data storage circuit 14 stores data of an image signal having undergone preprocessing as projection data.

The image processing circuit 16 executes image processing on data of a fluoroscopic image generated by the image generation circuit 15. This image processing includes, e.g., tone conversion processing for handling the contrast of a fluoroscopic image, smoothing processing for removing noise, and sharpening processing for emphasizing an edge. Of these image processes, image processing to be executed by the image processing circuit 16, and its parameters are decided by the user.

The input circuit 11 functions as an interface for inputting instruction information from the user to the X-ray diagnostic apparatus 1. The instruction information includes an instruction to move the top 5 and the C-arm 6, an instruction to start X-ray fluoroscopy, and an instruction to change the X-ray fluoroscopic direction. The input circuit 11 includes an operation console which is used by the user to move the top 5 and the C-arm 6 to desired positions. The operation console includes input devices such as a mouse, a keyboard, a track ball, a touch panel, and a switch. The input circuit 11 also includes a fluoroscopy switch for starting X-ray fluoroscopy. The fluoroscopy switch is constituted by, e.g., a foot switch. X-ray fluoroscopy is performed while the foot switch is pressed, and when the foot switch is released, the X-ray fluoroscopy is ended.

The display circuit 12 includes a monitor which is, e.g., an LCD (Liquid Crystal Display). The display circuit 12 displays an input window and a fluoroscopic image. The input window is a GUI (Graphical User Interface) for accepting an input from the user via the input circuit 11. The input window includes a fluoroscopy condition input window and the like for assisting input of X-ray fluoroscopy conditions by the user. A fluoroscopic image is input from the image generation circuit 15.

The imaging control circuit 17 performs ON/OFF control of X-ray irradiation, rotation/movement control of the C-arm 6, movement control of the top 5, and the like. More specifically, the imaging control circuit 17 controls the respective circuits in order to execute X-ray imaging in accordance with conditions concerning X-ray imaging, a timing designated by the user, and the like. More specifically, the imaging control circuit 17 starts control of the high voltage generator 2, the X-ray stop device 4, the X-ray detector 7, the C-arm driving mechanism 8, the top moving mechanism 9, the preprocessing circuit 13, the image generation circuit 15, and the like in response to pressing of the fluoroscopy switch by the user. The imaging control circuit 17 controls the high voltage generator 2 in accordance with X-ray apparatus conditions (tube current, tube voltage, and irradiation time) set by the user. At this time, the imaging control circuit 17 controls the X-ray detector 7, the preprocessing circuit 13, and the data storage circuit 14 together with the control of the high voltage generator 2, and stores projection data in the data storage circuit 14.

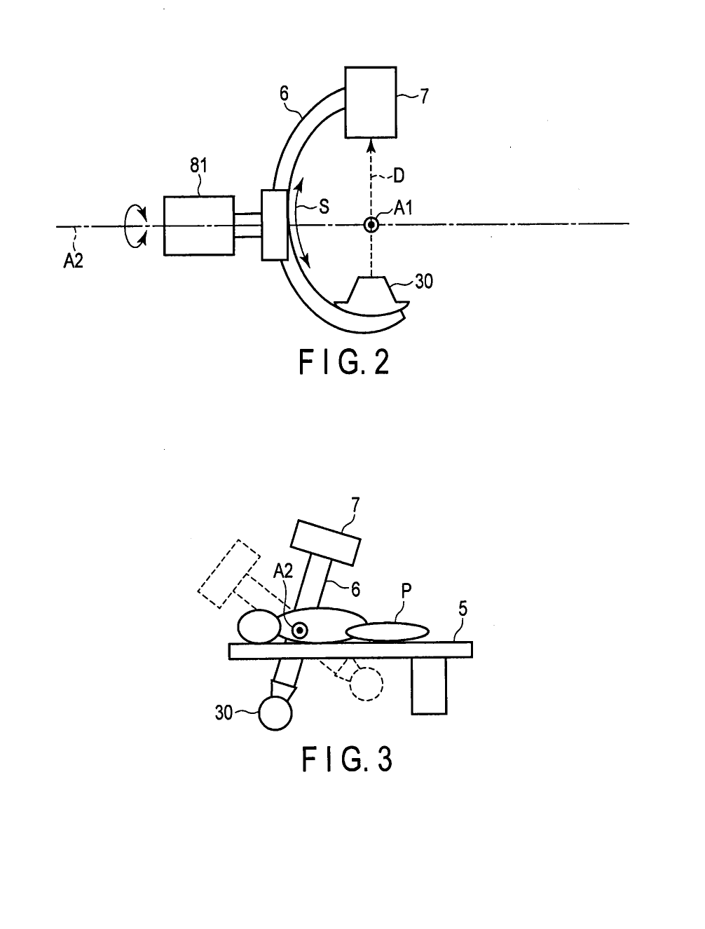

FIG. 2 is a schematic view for explaining rotation control of the C-arm 6 by the imaging control circuit 17. The C-arm driving mechanism 8 includes a C-arm holder 81 installed on the floor by a stand (not shown) so that the C-arm holder 81 can revolve or is stationary. The C-arm holder 81 supports the C-arm 6 so that the C-arm 6 can slide in a sliding direction S along the C shape. The C-arm 6 slides in the sliding direction S and rotates about a rotation axis A1. The C-arm holder 81 supports the C-arm 6 so that the C-arm 6 can rotate about a rotation axis A2 perpendicular to the rotation axis A1. The intersection point between the rotation axes A1 and A2 is called an isocenter.

Although not shown in FIG. 2, the C-arm driving mechanism 8 includes a vertically moving mechanism which moves the C-arm holder 81 and the C-arm 6 along a vertical axis in the direction of gravity, and a horizontally moving mechanism which moves the C-arm holder 81 and the C-arm 6 along a horizontal axis perpendicular to the vertical axis. Since multiaxis movement and multiaxis rotation of the C-arm 6 are possible in this manner, fluoroscopic images of the object P can be obtained from all directions in a three-dimensional space. Note that an arrow D shown in FIG. 2 indicates a direction from the X-ray source device 30 to the center of the X-ray detector 7 via the isocenter. In the following description, this direction will be called a projection direction. A straight line extending from the focus of the X-ray tube 3 to the center position of the X-ray detection surface of the X-ray detector 7 via the isocenter will be called an imaging center line.

The system control circuit 10 includes a CPU (Central Processing Circuit) and a semiconductor memory. The system control circuit 10 temporarily stores, in the semiconductor memory, information input to the X-ray diagnostic apparatus 1 via thee input circuit 11. The system control circuit 10 performs centralized control of the respective circuits of the X-ray diagnostic apparatus 1 based on the input information.

When performing treatment or diagnosis accompanied by insertion of a puncture needle, insertion of the puncture needle by the user is sometimes assisted by performing X-ray fluoroscopy. At this time, X-ray fluoroscopy is performed from two directions in which projection directions are perpendicular to each other. The first projection direction is a direction along the puncture needle insertion direction. The user views a fluoroscopic image corresponding to the first projection direction and can grasp whether the inserted puncture needle is inserted straight. The second projection direction is a direction perpendicular to the puncture needle insertion direction. The user views a projection image corresponding to the second projection direction and can grasp the length and position of the inserted puncture needle.

Problems when X-ray fluoroscopy is performed from the two directions perpendicular to each other in the above-described way will be explained.

FIG. 3 is a view for explaining problems at the time of inserting the puncture needle. FIG. 3 is a schematic view showing the states of the X-ray source device 30, X-ray detector 7, C-arm 6, top 5, and object P during treatment accompanied by insertion of the puncture needle. The X-ray source device 30, X-ray detector 7, and C-arm 6 indicated by solid lines represent a state in which they are arranged so that the projection direction becomes the first projection direction mentioned above. A case is assumed, in which the C-arm 6 is rotated from this state by, e.g., 90° about the rotation axis A2 so that the projection direction becomes the second projection direction. When the X-ray source device 30, the X-ray detector 7, and the C-arm 6 are rotated to positions indicated by broken lines during the 90° rotation, the X-ray source device 30 and the lower surface of the top 5 interfere with each other. In the rotation about the rotation axis A2, the object P cannot be imaged from the second projection direction. The 90° rotational operation by the C-arm 6 is repetitively performed during the puncture needle insertion work. Even if no interference occurs, the 90° rotational operation increases the time taken for treatment and the like, and increases the burden on a patient. In addition, the 90° rotational operation puts a heavy burden on mechanisms concerning the rotational operation, and may cause a trouble.

Embodiments of the X-ray diagnostic apparatus 1 including a means for solving these problems will be disclosed.

### First Embodiment

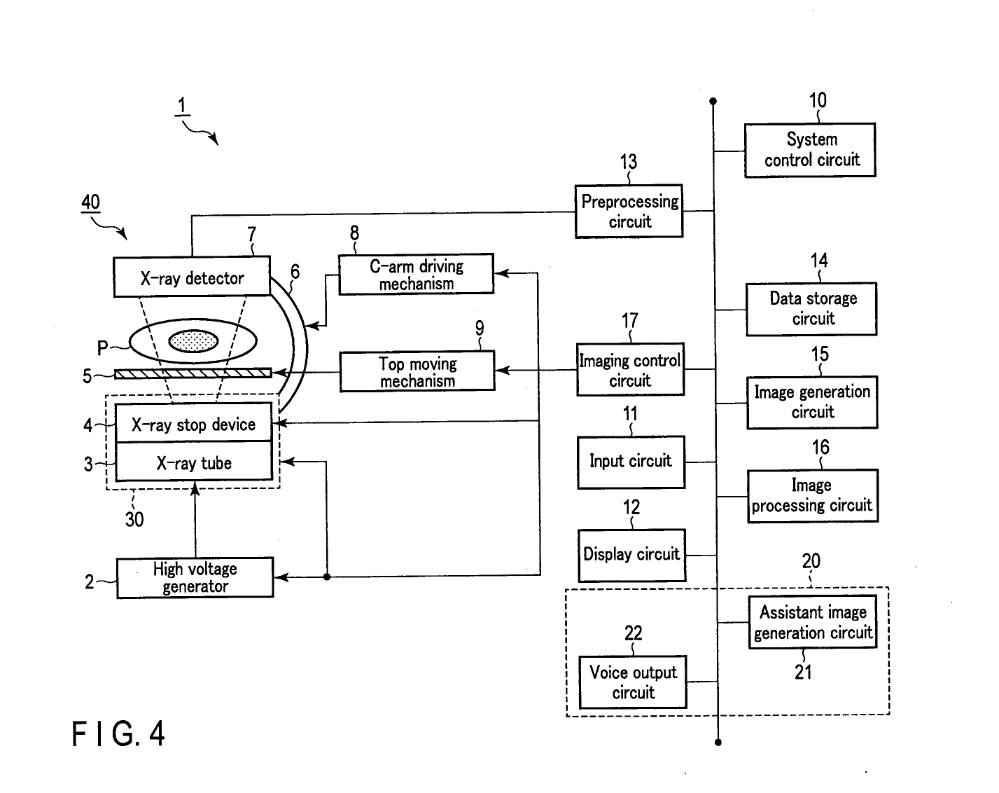

FIG. 4 is a block diagram of an X-ray diagnostic apparatus 1 according to the first embodiment. A description of repetitive contents will be omitted for the building components described with reference to FIG. 1.

An input circuit 11 includes three switches for inputting an X-ray fluoroscopic direction change instruction to the X-ray diagnostic apparatus 1. The three switches correspond to three projection modes different in projection direction. The first projection mode is a parallel mode in which the projection direction coincides with an initial planning direction. The input circuit 11 has a parallel switch for changing the projection mode to the parallel mode. The second projection mode is a tilt mode in which the projection direction is tilted by an angle θ smaller than 90° (0°<θ<90°) with respect to the parallel mode. The input circuit 11 has a tilt switch for changing the projection mode to the tilt mode. The third projection mode is a lateral mode in which the projection direction is perpendicular to that in the parallel mode. The input circuit 11 has a lateral switch for changing the projection mode to the lateral mode. These switches are used to change the projection direction. These switches may be mechanical switches or soft switches.

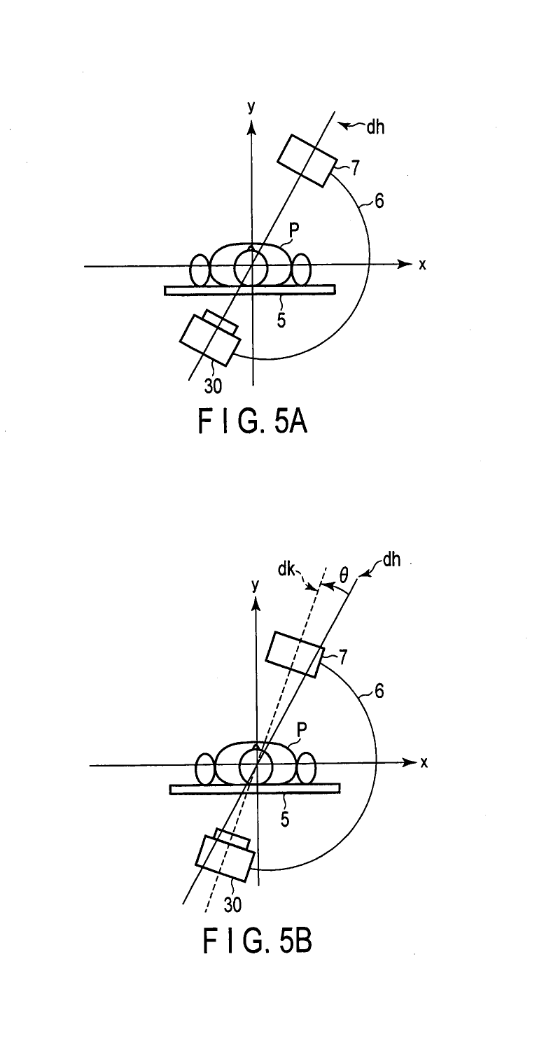

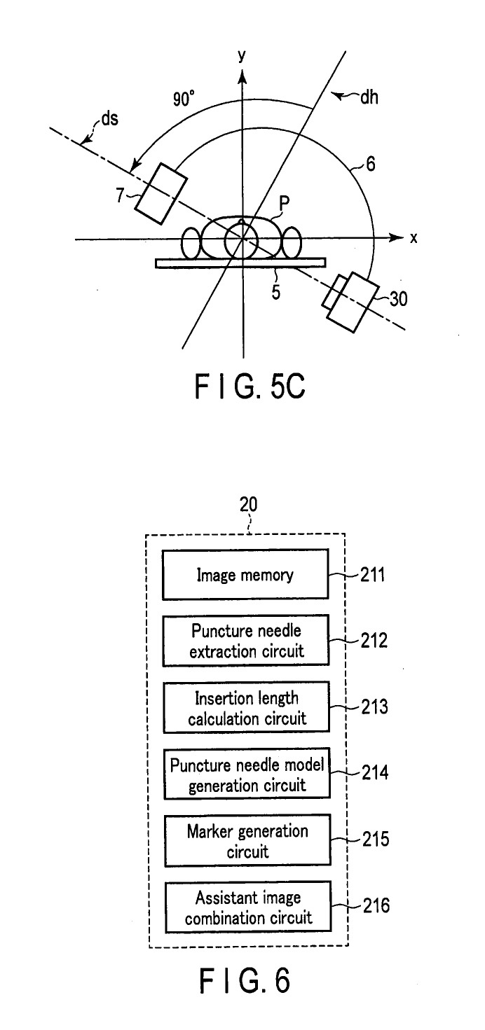

FIG. 5 is an explanatory view for explaining the three projection modes of the X-ray diagnostic apparatus 1 according to the first embodiment. The short-axis direction of a top 5 is defined as the x-axis, and the perpendicular direction (zenithal direction) of the top 5 is defined as the y-axis. FIG. 5 schematically shows the positional relationship between an imaging circuit 40 and the top 5 for each mode.

FIG. 5A is a schematic view showing the positional relationship between the imaging circuit 40 and an object P in the parallel mode.

As shown in FIG. 5A, in the parallel mode, an X-ray source device 30, X-ray detector 7, and C-arm 6, which constitute the imaging circuit 40, are arranged so that the projection direction coincides with the initial planning direction. A fluoroscopic image concerning the object P that is displayed on a display circuit 12 in the parallel mode corresponds to an image captured from the initial planning direction. An imaging center line in the parallel mode is indicated by an imaging center line dh.

FIG. 5B is a schematic view showing the positional relationship between the imaging circuit 40 and the object P in the tilt mode.

As shown in FIG. 5B, in the tilt mode, the X-ray source device 30, X-ray detector 7, and C-arm 6, which constitute the imaging circuit 40, are arranged so that the projection direction is tilted by the angle θ (0°<θ<90°) with respect to the initial planning direction. An imaging center line in the tilt mode is indicated by an imaging center line dk. That is, the imaging center lines dh and dk form the angle θ. Note that the angle θ can be an angle of, e.g., about 3° to 10°. The user can set and change the tilt angle θ via, e.g., the input circuit 11.

FIG. 5C is a schematic view showing the positional relationship between the imaging circuit 40 and the object P in the lateral mode.

As shown in FIG. 5C, in the lateral mode, the X-ray source device 30, X-ray detector 7, and C-arm 6, which constitute the imaging circuit 40, are arranged so that the projection direction is tilted by 90° with respect to the initial planning direction. An imaging center line in the lateral mode is indicated by an imaging center line dt. That is, the imaging center lines dh and dt form 90°.

The input circuit 11 inputs an insertion target position and an arrival target position to the X-ray diagnostic apparatus 1. These inputs are performed by user operations on an insertion condition setting window displayed on the display circuit 12. The insertion target position is set on the body surface of the object P. The insertion target position is a position at which a puncture needle is inserted into the object P. The arrival target position is set inside the object P. The arrival target position is a position at which the tip of the puncture needle is caused to arrive. The insertion condition input window includes a slice image concerning the object P, and an OK button. The slice image concerning the object P is generated by executing projection processing by an image processing circuit 16 on volume data concerning the object P. The volume data concerning the object P is stored in a data storage circuit 14. The OK button is a button for deciding information input by the user. These inputs are performed by a user operation via the input circuit 11. For example, the user can input an insertion target position by pointing a cursor to the insertion target position on the slice image with a mouse or the like, and then clicking the mouse or the like. By the same method, an arrival target position can be input. By clicking the OK button, the insertion target position and the arrival target position are set. The direction of a straight line (to be referred to as an insertion guideline hereinafter) connecting the set insertion target position and arrival target position will be called the initial planning direction.

The display circuit 12 displays an input window, a fluoroscopic image, and an assistant image. The input window is a GUI (Graphical User Interface) for accepting an input from the user via the input circuit 11. The input window includes a fluoroscopy condition input window for assisting input of X-ray fluoroscopy conditions by the user, and an insertion condition setting window for assisting input of an insertion target position and arrival target position by the user. The assistant image is input by an assistant image generation circuit 21 (to be described later). The display circuit 12 may display the assistant image over the fluoroscopic image, or switch and display the fluoroscopic image and the assistant image in accordance with a user instruction.

The data storage circuit 14 stores volume data concerning the object P. The volume data is collected in advance by the imaging circuit 40 before performing puncture into the object P. The imaging circuit 40 performs X-ray imaging of the object P while rotating around the object P under the control of an imaging control circuit 17. The volume data is three-dimensional image data reconstructed based on a plurality of projection data different in projection angle that have been collected by rotational imaging by the imaging circuit 40.

A notification circuit 20 notifies the user of assistant information which assists a puncture needle insertion operation. The notification circuit 20 notifies the user of assistant information by at least one of a voice and display. A notification method to be applied is set and changed in accordance with a user instruction via the input circuit 11.

The notification circuit 20 includes the assistant image generation circuit 21, and a voice output circuit 22 which outputs a voice.

The assistant image generation circuit 21 generates data of an assistant image for notifying the user of, as a display, assistant information which assists a puncture needle insertion operation. The generated assistant image data is output to the display circuit 12.

FIG. 6 is a block diagram showing the functional blocks of the assistant image generation circuit 21 according to the first embodiment. As shown in FIG. 6, the assistant image generation circuit 21 according to the first embodiment includes an image memory 211, a puncture needle extraction circuit 212, an insertion length calculation circuit 213, a puncture needle model generation circuit 214, a marker generation circuit 215, and an assistant image combination circuit 216.

The image memory 211 is a primary storage device which can be directly accessed by a system control circuit 10 (processor such as a CPU). The image memory 211 is a memory constituted by a semiconductor element and, for example, a DRAM (Dynamic Random Access Memory) is used for the primary storage device. Under the control of the system control circuit 10, the image memory 211 temporarily stores data to be handled by the notification circuit 20. The data to be handled by the notification circuit 20 is data of fluoroscopic images in the parallel mode and the tilt mode. Also, the image memory 211 stores data of an image concerning the object P that corresponds to a direction perpendicular to the initial planning direction. The image concerning the object P is data of an X-ray image that has been captured in accordance with a set initial planning direction before inserting the puncture needle. However, the image concerning the object P may be an image that has undergone projection processing by the image processing circuit 16 on volume data stored in the data storage circuit 14. Alternatively, the image concerning the object P may be one projection data out of a plurality of projection data different in projection angle that have been used to reconstruct volume data.

The puncture needle extraction circuit 212 extracts the region of the puncture needle included inside the object P from a fluoroscopic image. For example, threshold processing is applied to the extraction method.

The insertion length calculation circuit 213 specifies the apparent length of the puncture needle in the fluoroscopic image based on the region of the puncture needle extracted from a fluoroscopic image corresponding to the tilt node. Then, the insertion length calculation circuit 213 calculates the estimated insertion length of the puncture needle inserted into the object P based on the apparent length of the puncture needle, and the tilt angle θ in the tilt mode from the parallel mode. The insertion length calculation circuit 213 may calculate the ratio of the estimated insertion length to the target insertion length. The target insertion length is defined by the distance between the insertion target position and the arrival target position.

The puncture needle model generation circuit 214 generates data of the model image of the puncture needle corresponding to the estimated insertion length.

The voice output circuit 22 notifies the user of, as a sound or voice, assistant information which assists a puncture needle insertion operation. The voice output circuit 22 includes a loudspeaker (not shown). The voice output circuit 22 outputs, as a voice, text information corresponding to the estimated insertion length. In this case, the voice output circuit 22 outputs a voice such as “the current estimated insertion length is X mm.” or “the remaining length of insertion is Y mm.” from the loudspeaker. Text information corresponding to the ratio of the estimated insertion length to the target insertion length may be output as a voice. In this case, the voice output circuit 22 outputs a voice such as “the current insertion of the puncture needle is completed by 70%.” from the loudspeaker. At the timing when the ratio of the estimated insertion length to the target insertion length becomes equal to or higher than a threshold, the voice output circuit 22 may output, from the loudspeaker, a notification sound notifying the user that the puncture needle will soon arrive at the arrival target position.

The marker generation circuit 215 generates an insertion position marker indicting an insertion target position. Also, the marker generation circuit 215 generates an arrival position marker indicating an arrival target position. Note that the data storage circuit 14 may store data of a plurality of markers in advance, and the marker generation circuit 215 may select markers corresponding to the insertion position marker and arrival position marker from the plurality of markers.

The assistant image combination circuit 216 generates data of an assistant image by combining the model image of the puncture needle, the insertion position marker, and the arrival position marker with the image concerning the object P. More specifically, the assistant image combination circuit 216 arranges the insertion position marker at the insertion target position on the image concerning the object P, and arranges the arrival position marker at the arrival target position. The assistant image combination circuit 216 arranges the model image of the puncture needle in correspondence with the position of the puncture needle extracted by the puncture needle extraction circuit 212. The assistant image combination circuit 216 may arrange the insertion position marker and the arrival position marker on the fluoroscopic image.

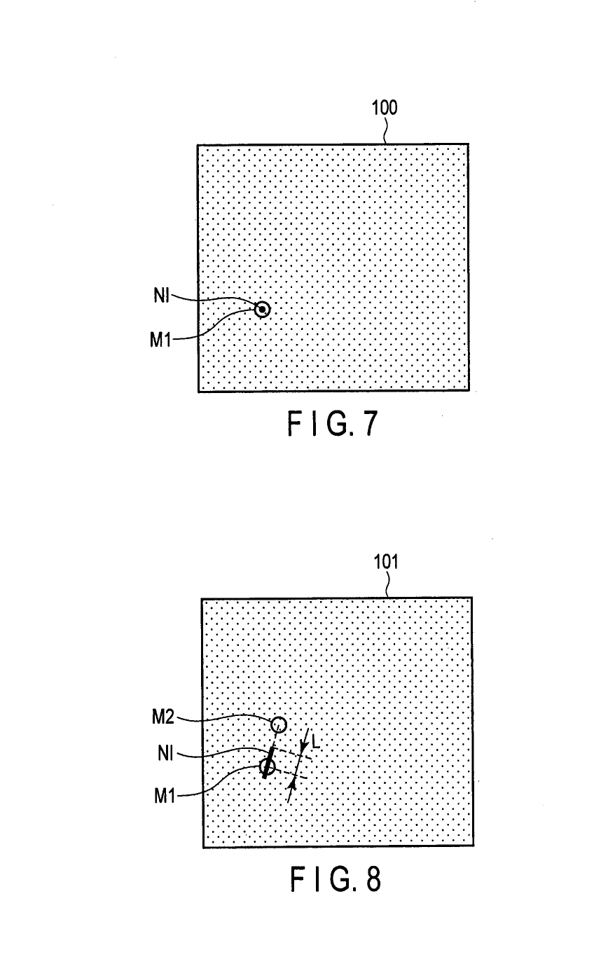

FIG. 7 is a view showing an example of a fluoroscopic image corresponding to the parallel mode according to the first embodiment. A fluoroscopic image 100 shown in FIG. 7 is captured from the initial planning direction. The fluoroscopic image 100 shown in FIG. 7 includes an insertion position marker M1 indicating an insertion target position. The doctor inserts the puncture needle at the insertion target position, and then puts the puncture needle straight toward an arrival target position while confirming the fluoroscopic image 100. The puncture needle is generally made of a material which absorbs X-rays well, and is drawn thickly in the fluoroscopic image 100. When the puncture needle is put straight from the insertion target position toward the arrival target position, a puncture needle image NI drawn in the fluoroscopic image 100 becomes a point.

FIG. 8 is a view showing an example of a fluoroscopic image corresponding to the tilt mode according to the first embodiment. A fluoroscopic image 101 shown in FIG. 8 is captured from a direction in which the projection direction is tilted by the angle θ (0°<θ<90°) with respect to the parallel mode. The fluoroscopic image 101 shown in FIG. 8 includes the insertion position marker M1 indicating an insertion target position, and an arrival position marker M2 indicating an arrival target position. In the fluoroscopic image 101 displayed in the tilt mode, the puncture needle image NI appears not as a point but as a straight line or curve. An apparent length L of the puncture needle will be described later.

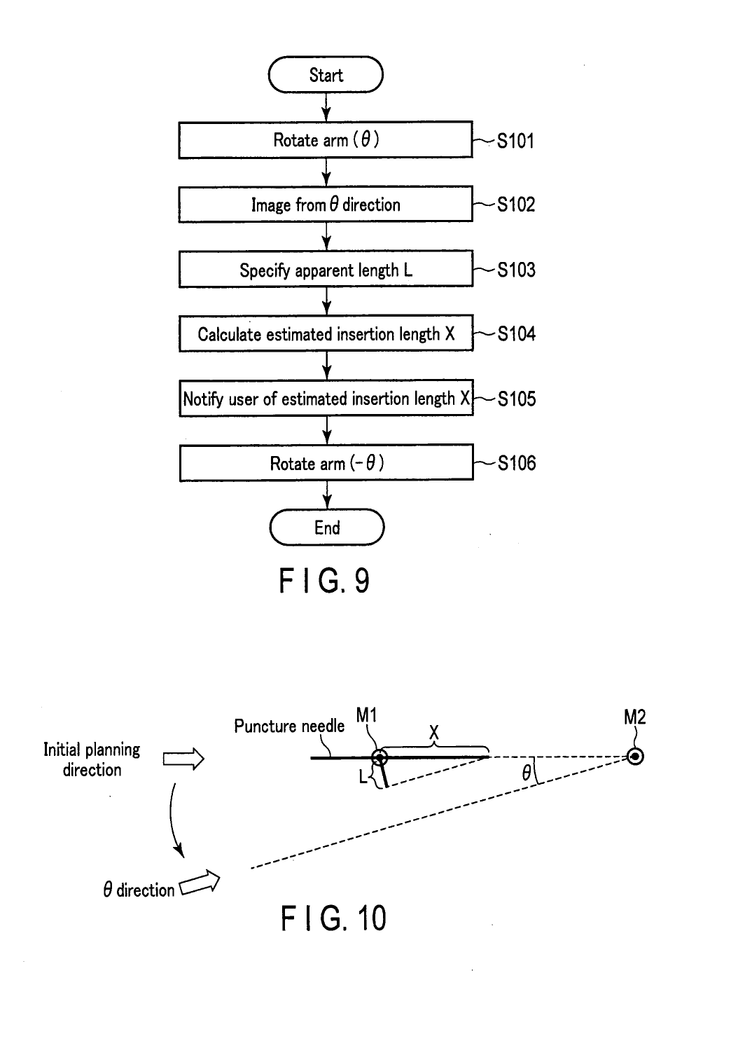

Next, procedures until the projection mode is switched from the parallel mode shown in FIG. 7 to the tilt mode shown in FIG. 8 and an assistant image is generated will be explained with reference to FIGS. 9 and 10.

FIG. 9 is a flowchart showing the operation of the X-ray diagnostic apparatus 1 upon switching from the parallel mode to the tilt mode.

First, when giving treatment accompanied by insertion of the puncture needle, the user such as a doctor inputs an insertion target position and an arrival target position on the insertion condition setting window. After setting the insertion target position and the arrival target position, the user presses the parallel switch. Then, the imaging control circuit 17 controls a C-arm driving mechanism 8 to move the C-arm 6 so as to set the parallel mode. At this time, the projection direction coincides with the initial planning direction. This movement includes rotation of the C-arm 6 and translation of the C-arm 6. In response to pressing of the fluoroscopy switch by the user, X-ray fluoroscopy of the object P is executed under the control of the imaging control circuit 17. As a result, a fluoroscopic image from the initial planning direction can be obtained. The display circuit 12 displays, as a moving image, the fluoroscopic image obtained by X-ray fluoroscopy (see FIG. 6).

When the user wants to confirm the length of the puncture needle inserted into the object P, he presses the tilt switch of the input circuit 11. In response to this, the projection mode is switched from the parallel mode to the tilt mode. Then, the X-ray diagnostic apparatus 1 operates according to the flowchart shown in FIG. 9.

In this flowchart, first, the imaging control circuit 17 controls the C-arm driving mechanism 8 to rotate the C-arm 6 by the angle θ (step S101). Accordingly, the projection mode is switched from the parallel mode to the tilt mode. At this time, the tilt direction may be set in advance or decided in accordance with a user instruction. The axis of this rotation can be a predetermined one of the rotation axes A1 and A2. Alternatively, the axis of this rotation can be an axis different from both the rotation axes A1 and A2. By combining rotational operations about the rotation axes A1 and A2, a rotation about these axes becomes possible. By this rotation, the projection direction is tilted by the angle θ with respect to the initial planning direction. In the following description, this projection direction will be called a θ direction.

After step S101, the imaging control circuit 17 controls the respective circuits of the imaging circuit 40 to capture a fluoroscopic image from the θ direction in the current state of the C-arm 6 (step S102). The imaging control circuit 17 may automatically start X-ray fluoroscopy after arranging the imaging circuit 40 at a position corresponding to the tilt mode, or may start X-ray fluoroscopy in response to pressing of the fluoroscopy switch. These settings can be appropriately changed in accordance with a user instruction via the input circuit 11. The imaging control circuit 17 displays the fluoroscopic image on the display circuit 12 (see FIG. 7).

After step S102, the puncture needle extraction circuit 212 extracts the region of the puncture needle image NI included inside the object P from the fluoroscopic image 101 captured in step S102. Based on the extracted region of the puncture needle image NI, the insertion length calculation circuit 213 specifies the apparent length L of the puncture needle in the fluoroscopic image (step S103). More specifically, the apparent length L of the puncture needle is the length of the puncture needle image NI between the markers M1 and M2 in the fluoroscopic image captured in step S102. The specified apparent length L of the puncture needle is, e.g., a numerical value obtained by multiplying a length indicated by pixels in the fluoroscopic image 101, by a coefficient for converting one pixel into a length in a real space.

After step S103, the insertion length calculation circuit 213 calculates an estimated insertion length X (mm) of the puncture needle inserted in the object P (step S104). A method of calculating the estimated insertion length X will be explained with reference to FIG. 9.

FIG. 10 is an explanatory view for explaining a method of calculating an estimated insertion length. FIG. 10 is associated with FIG. 8. The insertion position marker M1 indicates the insertion target position, and the arrival position marker M2 indicates the arrival target position. As is geometrically apparent from this drawing, the estimated insertion length X is obtained by:

??

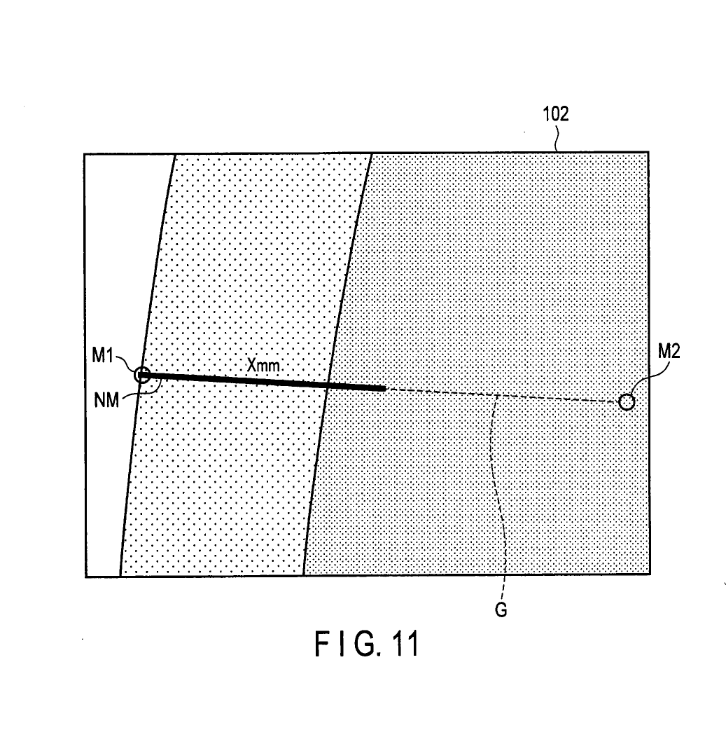

That is, based on the apparent length L of the puncture needle and the tilt angle θ in the tilt mode from the parallel mode, the insertion length calculation circuit 213 can calculate the estimated insertion length X of the puncture needle currently inserted in the object P. After step S104, the notification circuit 20 notifies the user of the estimated insertion length X calculated in step S104 (step S105). For example, the notification circuit 20 gives this notification by using an assistant image 102 as shown in FIG. 11 that is displayed on the display circuit 12.

FIG. 11 is a view showing an example of the assistant image generated by the assistant image generation circuit 21 according to the first embodiment. The assistant image combination circuit 216 generates the assistant image 102. The assistant image 102 is an image obtained by combining a model image NM of the puncture needle, the insertion position marker M1, and the arrival position marker M2 with the image concerning the object P. More specifically, the assistant image combination circuit 216 arranges the insertion position marker M1 at the insertion target position on the image concerning the object P, and arranges the arrival position marker M2 at the arrival target position. The assistant image combination circuit 216 arranges the model image NM of the puncture needle in correspondence with the position of the puncture needle image NI extracted by the puncture needle extraction circuit 212. The length of the model image NM of the puncture needle corresponds to the estimated insertion length. The assistant image 102 includes an insertion guideline G connecting the insertion position marker M1 and the arrival position marker M2, and a character string “Xmm” representing the estimated insertion length X. For example, when the puncture needle is inserted straight from the insertion target position toward the arrival target position, the model image NM of the puncture needle overlaps the insertion guideline G, as shown in FIG. 11.

The assistant image 102 is displayed in a preset layout on the display circuit 12. The layout can be properly changed in accordance with a user instruction via the input circuit 11. For example, the assistant image 102 is displayed parallel to the fluoroscopic image. The assistant image 102 may be displayed on the fluoroscopic image at a size smaller than that of the fluoroscopic image. The assistant image 102 may be displayed on the display circuit 12 when, for example, an insertion target position and an arrival target position are set, or displayed on the display circuit 12 every time step S105 is executed. In this case, after executing step S106 (to be described later), the assistant image 102 disappears.

After step S105, the imaging control circuit 17 controls the C-arm driving mechanism 8 to rotate the C-arm 6 by an angle −θ in response to pressing of the parallel switch (step S106). The axis of this rotation is the same as that in step S101. That is, by this rotation, the projection mode is switched from the tilt mode to the parallel mode, and the projection direction coincides with the initial planning direction.

After step S106, the X-ray diagnostic apparatus 1 ends the processing shown in this flowchart. Thereafter, the X-ray diagnostic apparatus 1 restarts X-ray fluoroscopy in the parallel mode, and a fluoroscopic image corresponding to the parallel mode is displayed on the display circuit 12. Note that the imaging control circuit 17 may automatically start X-ray fluoroscopy after the imaging circuit 40 is returned to a position corresponding to the parallel mode, or start X-ray fluoroscopy in response to pressing of the fluoroscopy switch.

The X-ray diagnostic apparatus 1 according to the above-described embodiment rotates the C-arm 6 by only the angle θ smaller than 90° from the initial planning direction, and can estimate the length X of the puncture needle currently inserted into the object P. The X-ray diagnostic apparatus 1 can notify the user of the estimated puncture needle length X by the display of an assistant image or a voice. The X-ray source device 30, the X-ray detector 7, or the C-arm 6 hardly interferes with the top 5 or the object P, and the time taken till the completion of rotation is shortened, compared to a case in which the C-arm 6 is repetitively rotated in two directions perpendicular to each other in the puncture needle insertion operation.

Various other preferable effects are obtained from the arrangement disclosed in this embodiment.

### Second Embodiment

The second embodiment assumes a case in which the insertion direction of a puncture needle inserted in an object P shifts from an insertion guideline. An X-ray diagnostic apparatus 1 according to the second embodiment will be explained below mainly for a difference from the first embodiment.

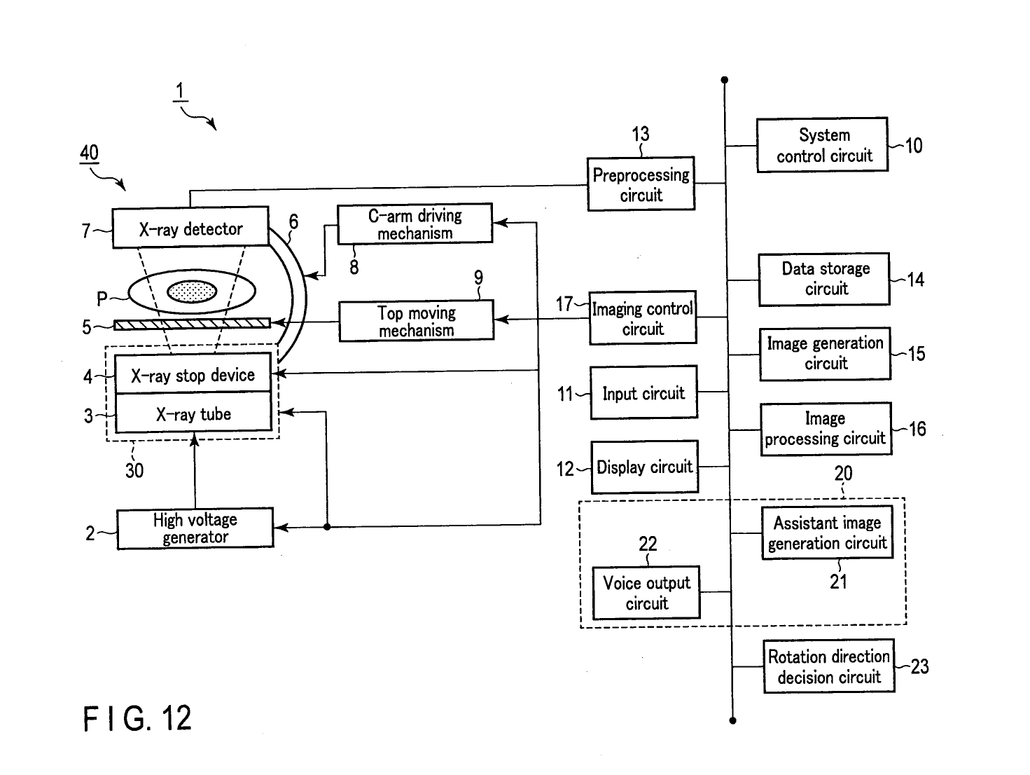

FIG. 12 is a block diagram showing the X-ray diagnostic apparatus 1 according to the second embodiment. A description of repetitive contents will be omitted for the building components described with reference to FIGS. 1 and 4.

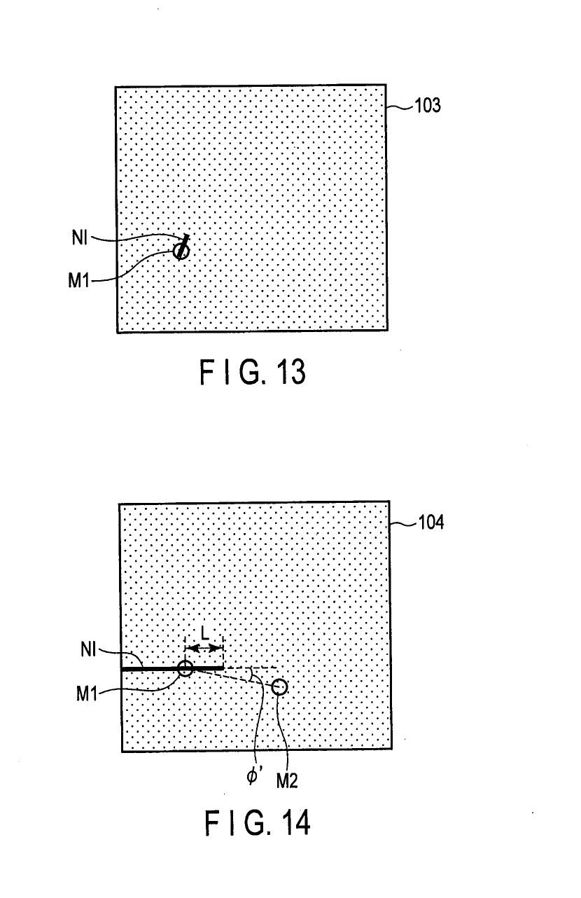

FIG. 13 is a view showing an example of a fluoroscopic image corresponding to the parallel mode according to the second embodiment. A fluoroscopic image 103 shown in FIG. 13 is captured from the initial planning direction. The fluoroscopic image 103 shown in FIG. 13 includes an insertion position marker M1 indicating an insertion target position. Since the second embodiment assumes that the puncture needle insertion direction shifts from the insertion guideline, a puncture needle image NI is represented by not a point but by a straight line in the fluoroscopic image 103. Note that when a soft puncture needle is used, the puncture needle image NI may be not a straight line but a curve. The user views the fluoroscopic image 103 and can confirm that the puncture needle is not inserted straight from the insertion target position to the arrival target position.

FIG. 14 is a view showing an example of a fluoroscopic image corresponding to the tilt mode according to the second embodiment. A fluoroscopic image 104 shown in FIG. 14 is captured from a direction in which the projection direction is tilted by the angle θ (0°<θ<90°) with respect to the projection direction in the parallel mode. The fluoroscopic image 104 shown in FIG. 14 includes the insertion position marker M1 indicating an insertion target position, and an arrival position marker M2 indicating an arrival target position. In the fluoroscopic image 104 displayed in the tilt mode, the puncture needle image NI is represented not by a point but by a straight line or curve. An apparent length L and shift angle φ′ of the puncture needle inside the object P will be described later.

A rotation direction decision circuit 23 decides the rotation direction of a C-arm 6 when the projection mode is switched from the parallel mode to the tilt mode or from the parallel mode to the lateral mode.

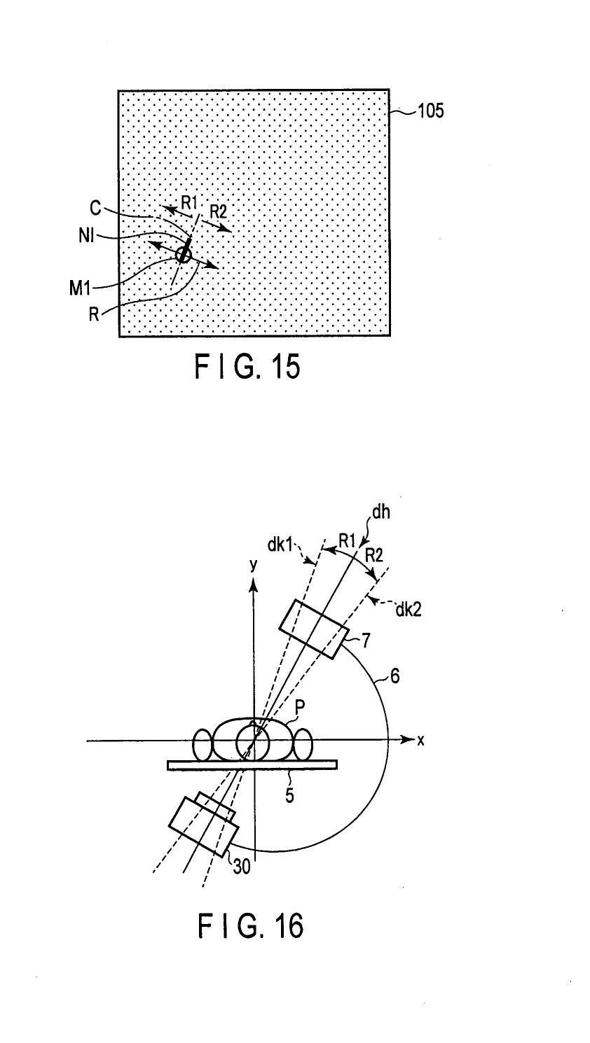

FIG. 15 is an explanatory view for explaining a method of deciding a rotation direction by the rotation direction decision circuit 23. FIG. 15 is a view showing a center axis C of the puncture needle image NI and a rotation direction R in a fluoroscopic image 105 shown in FIG. 13. The rotation direction decision circuit 23 obtains the center axis C of the puncture needle image NI. The center axis C is, e.g., an approximate straight line whose elements are pixels constituting the puncture needle image NI. In other words, the puncture needle image NI has an elliptical or rectangular shape in the second embodiment. Hence, the center axis C is the major axis of the ellipse or the major axis of the rectangle. The rotation direction decision circuit 23 decides a direction perpendicular to the center axis C on the fluoroscopic image 105 as the rotation direction R. As a result, the rotation axis of the C-arm 6 is decided. The rotation direction decision circuit 23 decides a direction R1 or a direction R2 as the tilt direction of the C-arm 6 in accordance with a user instruction. Note that the rotation direction decision circuit 23 may automatically decide the tilt angle in accordance with the positional relationship between an imaging circuit 40 and a top 5 in the parallel mode.

FIG. 16 is an explanatory view for explaining a method of deciding the tilt direction of the C-arm 6 by the rotation direction decision circuit 23. In FIG. 16, assume that the user presses the tilt switch, and the projection mode is switched from the parallel mode to the tilt mode. FIG. 16 schematically shows the positional relationship between the imaging circuit 40 and the top 5 in the parallel mode. The short-axis direction of the top 5 is defined as the x-axis, and the perpendicular direction (zenithal direction) of the top 5 is defined as the y-axis. In FIG. 16, the directions R1 and R2 correspond to the directions R1 and R2 described with reference to FIG. 15, respectively. An imaging center line dh represents the imaging center line of the imaging circuit 40 in the parallel mode. An imaging center line dk1 represents the imaging center line of the imaging circuit 40 when the C-arm 6 is tilted by the angle θ in the direction R1 at the time of switching the projection mode from the parallel mode to the tilt mode. An imaging center line dk2 represents the imaging center line of the imaging circuit 40 when the C-arm 6 is tilted by the angle θ in the direction R2 at the time of switching the projection mode from the parallel mode to the tilt mode.

The rotation direction decision circuit 23 decides the tilt direction of the C-arm 6 so as to minimize the angle formed by an imaging center line after tilting and the y-axis (zenithal direction) out of a plurality of candidates of the tilt direction. As shown in FIG. 16, the imaging center line dk1 out of the imaging center lines dk1 and dk2 is an imaging center line that minimizes the angle formed together with the y-axis (zenithal direction). Thus, the rotation direction decision circuit 23 decides the direction R1 as the tilt direction of the C-arm 6 when the projection mode is switched from the parallel mode to the tilt mode.

In general, a state in which the imaging circuit 40 is arranged so that the imaging center line coincides with the zenithal direction is an initial position. In the state in which the imaging circuit 40 is arranged at the initial position, the risk of interference of another mechanism, the object P, and the user with the imaging circuit 40 is low. This risk can be reduced by deciding the tilt angle of the C-arm 6 so as to minimize the angle formed by the imaging center line and the zenithal direction when the projection mode is switched from the parallel mode to another mode. Note that the tilt direction decision processing by the rotation direction decision circuit 23 is also applicable to the first embodiment. That is, the rotation direction decision circuit 23 may be included in the X-ray diagnostic apparatus 1 according to the first embodiment, and decide the tilt direction of the C-arm 6 so as to minimize the angle formed by the imaging center line and an axis in the zenithal direction in the tilt mode in response to pressing of the tilt switch.

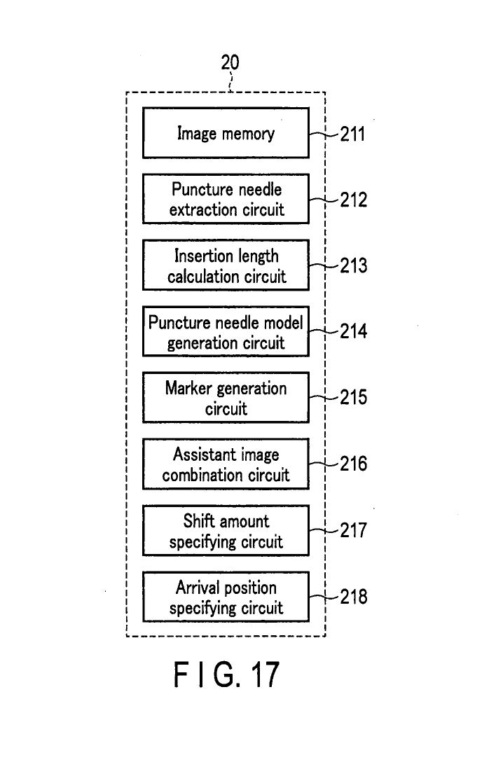

FIG. 17 is a block diagram showing the functional blocks of an assistant image generation circuit 21 according to the second embodiment. As shown in FIG. 17, the assistant image generation circuit 21 according to the second embodiment includes an image memory 211, a puncture needle extraction circuit 212, an insertion length calculation circuit 213, a puncture needle model generation circuit 214, a marker generation circuit 215, an assistant image combination circuit 216, a shift amount specifying circuit 217, and an arrival position specifying circuit 218.

The shift amount specifying circuit 217 specifies an insertion shift angle φ based on a fluoroscopic image corresponding to the tilt mode. The insertion shift angle φ is an angle formed by the initial planning direction and the puncture needle insertion direction. That is, the shift angle φ indicates an angle by which the puncture needle, which should be inserted straight from an insertion target position to an arrival target position, is inserted with a shift. Procedures to specify the insertion shift angle φ will be explained with reference to the fluoroscopic image 104 shown in FIG. 14. The shift amount specifying circuit 217 specifies an angle φ′ formed by the center axis of the puncture needle image NI and an insertion guideline connecting the insertion position marker M1 and the arrival position marker M2. The angle φ′ indicates an apparent shift angle in the fluoroscopic image. As is geometrically apparent, the apparent shift angle φ′ comes close to the insertion shift angle φ as the angle θ (0°<θ<90°) comes close to 90°, and the apparent shift angle φ′ and the insertion shift angle φ become more different as the angle θ decreases. The shift amount specifying circuit 217 specifies the insertion shift angle φ based on the apparent shift angle φ′ and the angle θ. The shift amount specifying circuit 217 holds a predetermined function in order to specify the insertion shift angle φ.

The arrival position specifying circuit 218 specifies the arrival prospective position of the puncture needle based on the insertion shift angle φ. The arrival prospective position is represented by a coordinate system on the assistant image. The arrival prospective position indicates, e.g., a position at which the tip of the puncture needle arrives when the puncture needle proceeds at the current insertion angle. A method of specifying an arrival prospective position by the arrival position specifying circuit 218 will be described later.

The marker generation circuit 215 generates a prospective position marker indicating an arrival prospective position. A data storage circuit 14 may store in advance data of a plurality of markers, and the marker generation circuit 215 may select a prospective position marker from the plurality of markers.

The assistant image combination circuit 216 generates data of an assistant image by combining the model image of the puncture needle, the insertion position marker, the arrival position marker, and the prospective position marker with the image concerning the object P. More specifically, the assistant image combination circuit 216 arranges the insertion position marker at the insertion target position on the image concerning the object P, arranges the arrival position marker at the arrival target position, and arranges the prospective position marker at the arrival prospective position. The assistant image combination circuit 216 arranges the model image of the puncture needle in correspondence with the position of the puncture needle extracted by the puncture needle extraction circuit 212. The assistant image combination circuit 216 may arrange the insertion position marker, the arrival position marker, and the prospective position marker on the fluoroscopic image.

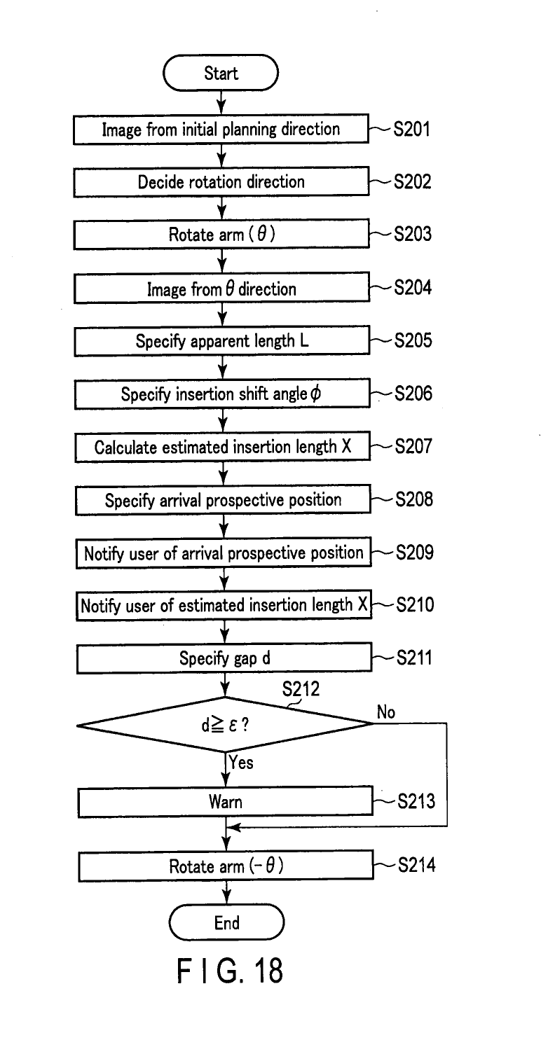

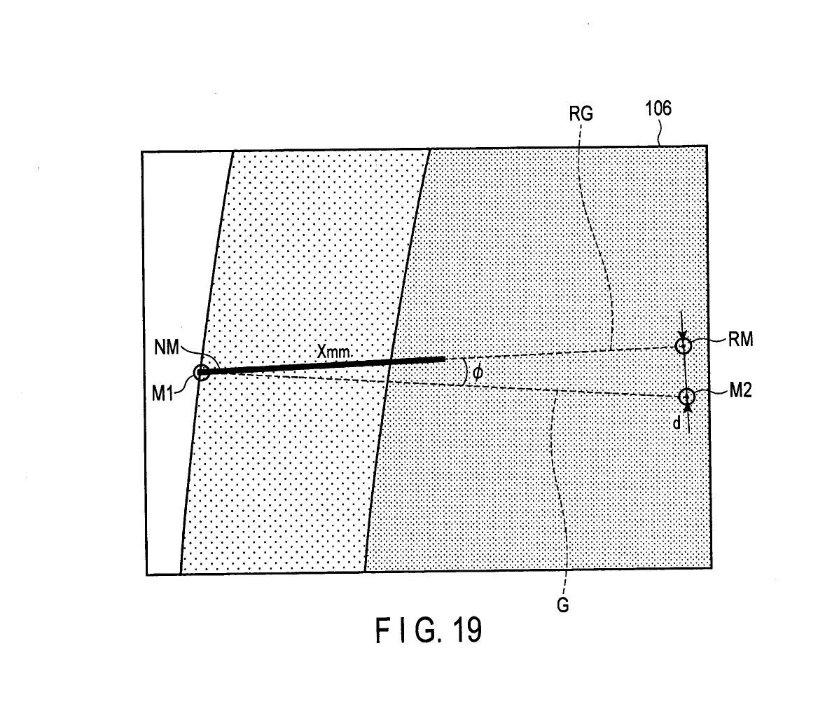

Next, procedures until the projection mode is switched from the parallel mode to the tilt mode, an assistant image is displayed, and the projection mode is returned from the tilt mode to the parallel mode will be explained with reference to FIGS. 18 and 19.

FIG. 18 is a flowchart showing an example of the operation of the X-ray diagnostic apparatus 1 according to the second embodiment. The flowchart of FIG. 18 shows procedures until the projection mode is switched from the parallel mode to the tilt mode, an assistant image is displayed, and the projection mode is returned from the tilt mode to the parallel mode.

When giving treatment accompanied by insertion of the puncture needle, an insertion target position and an arrival target position are set, and a fluoroscopic image corresponding to the initial planning direction is displayed on a display circuit 12, as in the first embodiment.

When the user wants to confirm the length of the puncture needle inserted into the object P and the position of the puncture needle with respect to the insertion guideline, he presses the tilt switch of an input circuit 11. Then, the projection mode is switched from the parallel mode to the tilt mode. In response to this, the X-ray diagnostic apparatus 1 operates according to the flowchart shown in FIG. 11.

In this flowchart, first, an imaging control circuit 17 controls the imaging circuit 40 to capture a fluoroscopic image from the initial planning direction (step S201). By this imaging, a fluoroscopic image as shown in FIG. 13 is displayed on the display circuit 12.

After step S201, the imaging control circuit 17 decides the rotation direction and tilt direction of the C-arm 6 based on the fluoroscopic image captured in step S201 (step S202).

After step S202, the imaging control circuit 17 controls a C-arm driving mechanism 8 to rotate the C-arm 6 by the angle θ in the decided direction about an axis corresponding to the rotation direction decided in step S202 (step S203). Accordingly, the projection mode is switched from the parallel mode to the tilt mode. The rotation axis in this case is, e.g., an axis which passes through the isocenter and is parallel to the above-mentioned center axis C.

After step S203, the imaging control circuit 17 controls the respective circuits of the imaging circuit 40 to capture a fluoroscopic image from the θ direction in the current state of the C-arm 6 (step S204). The imaging control circuit 17 may automatically start X-ray fluoroscopy after arranging the imaging circuit 40 at a position corresponding to the tilt mode, or may start X-ray fluoroscopy in response to pressing of the fluoroscopy switch. These settings can be appropriately changed in accordance with a user instruction via the input circuit 11. The imaging control circuit 17 displays the fluoroscopic image on the display circuit 12. By this imaging, a fluoroscopic image as shown in FIG. 14 is displayed on the display circuit 12.

After step S204, the puncture needle extraction circuit 212 extracts the region of the puncture needle image NI included inside the object P from the fluoroscopic image captured in step S205. Based on the extracted region of the puncture needle image NI, the insertion length calculation circuit 213 specifies the apparent length L of the puncture needle in the fluoroscopic image (step S205). A method of specifying the apparent length L is the same as that in step S103.

After step S205, the shift amount specifying circuit 217 specifies the insertion shift angle φ based on the fluoroscopic image captured in step S204 (step S206).

After step S206, the insertion length calculation circuit 213 calculates an estimated insertion length X (mm) of the puncture needle currently inserted in the object P (step S207). A method of calculating the estimated insertion length X is the same as that in step S104. However, when the estimated insertion length X is calculated by the method in step S104, an error arises from the insertion shift angle φ. The insertion length calculation circuit 213 may correct this error by, for example, multiplying the estimated insertion length X by a coefficient corresponding to the insertion shift angle φ. This coefficient can be set in advance based on the geometrical relationship between the apparent length L, the estimated insertion length X, the angle θ, and the insertion shift angle φ.

After step S207, the arrival position specifying circuit 218 specifies the arrival prospective position of the puncture needle based on the insertion shift angle φ specified in step 206 (step S208).

After step S208, a notification circuit 20 notifies the user of the arrival prospective position specified in step S208 (step S209). Further, the notification circuit 20 notifies the user of the estimated insertion length X calculated by the insertion length calculation circuit 213 in step S207 (step S210).

The notification circuit 20 gives this notification by using an assistant image 105 as shown in FIG. 19 that is displayed on the display circuit 12.

FIG. 19 is a view showing an example of the assistant image generated by the assistant image generation circuit 21 according to the second embodiment. The assistant image combination circuit 216 generates an assistant image 106. The assistant image 106 is an image obtained by combining a model image NM of the puncture needle, the insertion position marker M1, the arrival position marker M2, and a prospective position marker RM with the image concerning the object P. More specifically, the assistant image combination circuit 216 arranges the insertion position marker M1 at the insertion target position on the image concerning the object P, arranges the arrival position marker M2 at the arrival target position, and arranges the prospective position marker RM at the arrival prospective position. The distance between the insertion position marker M1 and the prospective position marker RM is equal to the distance (target insertion length) between the insertion position marker M1 and the arrival position marker M2. That is, the arrival position specifying circuit 218 specifies the arrival prospective position based on the insertion shift angle θ, the target insertion, and the like. The assistant image combination circuit 216 arranges the model image NM of the puncture needle in accordance with the insertion shift angle θ. The length of the model image NM of the puncture needle corresponds to the estimated insertion length. The assistant image 106 includes an insertion guideline G connecting the insertion position marker M1 and the arrival position marker M2, and a character string “Xmm” representing the estimated insertion length X. The assistant image 106 may include a character string representing the angle φ formed by the insertion guideline G and an actual insertion line RG. The actual insertion line RG is a straight line connecting the insertion position marker M1 and the prospective position marker RM. The length of the model image NM of the puncture needle corresponds to a length obtained by multiplying the estimated insertion length X by a coefficient for converting the actual length of the puncture needle into a length on the assistant image 106. A gap d will be explained in the next step.

After step S210, the shift amount specifying circuit 217 specifies the gap d between the arrival position marker M2 and the prospective position marker RM (step S211). The gap d is a numerical value obtained by, for example, multiplying the length, indicated by pixels, of a straight line connecting the arrival position marker M2 and the prospective position marker RM, by a coefficient for converting one pixel into a length in a real space. The distance between these two positions may be calculated based on the coordinates of the arrival target position on the fluoroscopic image 105 and those of the arrival prospective position, and may be defined as the gap d. The gap d may be a distance from the prospective position marker to the insertion guideline G. In this case, the distance from the prospective position marker RM to the insertion guideline G can be calculated using a trigonometric function based on the target insertion length and the insertion shift angle 4.

After step S211, the notification circuit 20 determines whether the gap d is equal to or larger than a predetermined threshold ε (step S212). The threshold ε is a distance for isolating a case in which the puncture needle insertion operation needs to be retried, and a case in which insertion of the puncture needle can be continued. As the threshold ε, different values may be set for every puncture purpose, every puncture target region, and every user. The concrete value of the threshold ε can be set theoretically or empirically.

If the notification circuit 20 determines that the gap d is equal to or larger than the predetermined threshold ε (YES in step S212), it generates a warning (step S213). This warning is given by, for example, displaying on the display circuit 12 a message that the puncture work needs to be retried. In addition, for example, the voice output circuit 22 may output from a loudspeaker a voice or sound corresponding to a predetermined warning. The voice or sound output from the loudspeaker is arbitrary as long as the user can recognize the warning. The assistant image combination circuit 216 may generate an assistant image so that the model image NM of the puncture needle flickers on the assistant image 106. Note that the gap d is used as a distance for isolating a case in which the puncture needle insertion operation needs to be retried, and a case in which insertion of the puncture needle can be continued. However, another parameter may be used. For example, the notification circuit 20 may generate a warning when the insertion shift angle φ is equal to or larger than a threshold angle η.

After step S213, or if the notification circuit 20 determines that the gap d is smaller than the threshold ε (NO in step S212), the imaging control circuit 17 controls the C-arm driving mechanism 8 to rotate the C-arm 6 by an angle −θ in response to pressing of the parallel switch (step S214). The axis of this rotation is the same as that in step S203. That is, by this rotation, the projection mode is switched from the tilt mode to the parallel mode, and the projection direction coincides with the initial planning direction.

After step S214, the X-ray diagnostic apparatus 1 ends the processing shown in this flowchart. Thereafter, the X-ray diagnostic apparatus 1 restarts X-ray fluoroscopy in the parallel mode, and a fluoroscopic image corresponding to the parallel mode is displayed on the display circuit 12. The imaging control circuit 17 may automatically start X-ray fluoroscopy after the imaging circuit 40 is arranged at a position corresponding to the parallel mode, or start X-ray fluoroscopy in response to pressing of the fluoroscopy switch.

The above-described embodiment can obtain the following effects, in addition to the same effects as those in the first embodiment.

By referring to the assistant image 106 as shown in FIG. 19, the user can easily know the degree of shift by which the current insertion direction of the puncture needle shifts from the initial planning direction.

Further, by referring to the assistant image 106, the user can easily know an arrival prospective position obtained when the current insertion of the puncture needle proceeds.

Since a warning is generated when the gap between the arrival target position and the arrival prospective position is large, the user can quickly determine whether to retry the puncture.

Various other preferable effects are obtained from the arrangement disclosed in this embodiment.

The flowcharts described in the first and second embodiments show procedures concerning switching from the parallel mode to the tilt mode. However, in the tilt mode, the user can confirm not the actual insertion length of the puncture needle but an insertion length of the puncture needle that is estimated based on a fluoroscopic image corresponding to the tilt mode. This is because the projection direction in the tilt mode is a direction tilted by an angle θ (0°<θ<90°) of smaller than 90° from the initial planning direction. Hence, the user needs to confirm the actual insertion length of the puncture needle. That is, a workflow is preferable, in which the user performs puncture needle insertion work in the parallel mode, simply confirms the insertion length and insertion position of the puncture needle in the tilt mode, and confirms in detail the insertion length and insertion position of the puncture needle in the lateral mode. A series of workflow operations using the X-ray diagnostic apparatus 1 according to the second embodiment will be explained below. Note that this workflow is also applicable to the X-ray diagnostic apparatus 1 according to the first embodiment.

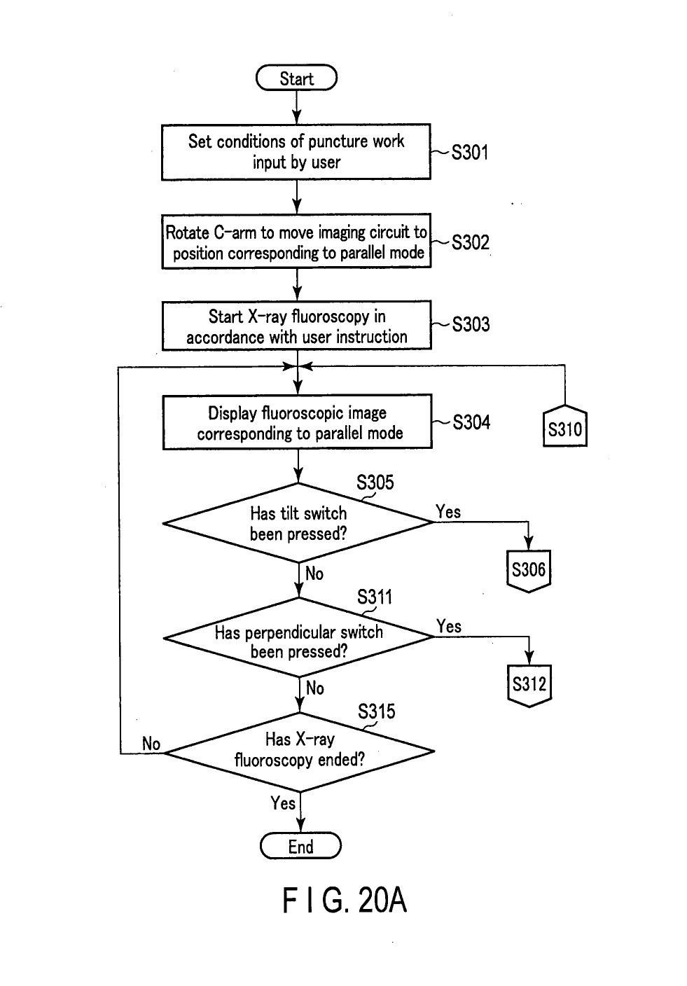

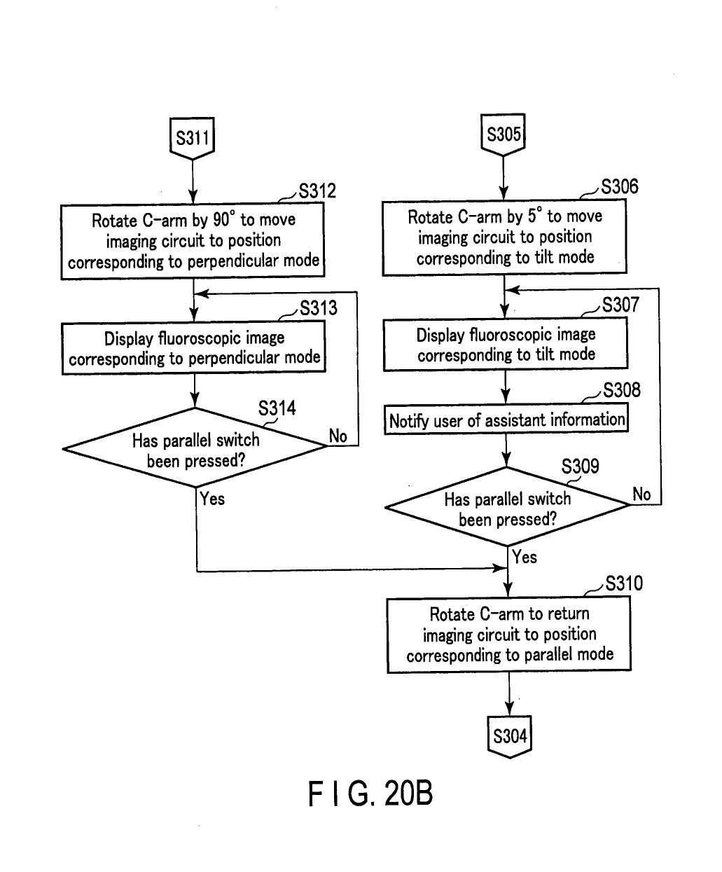

FIG. 20A is a flowchart showing part of the workflow of the first example using the X-ray diagnostic apparatus 1 according to the second embodiment.

FIG. 20B is a flowchart showing a remaining part of the workflow of the first example using the X-ray diagnostic apparatus 1 according to the second embodiment.

First, when giving treatment accompanied by insertion of the puncture needle, the user such as a doctor sets conditions concerning puncture work (step S301). These conditions are, e.g., an insertion target position, an arrival target position, a tilt angle θ, and an assistant information notification method. Here, the tilt angle θ is “5°”, and the assistant information notification method is “display of an assistant image”. After setting the conditions, the user presses the parallel switch. Then, the imaging control circuit 17 controls the C-arm driving mechanism 8 to move the C-arm 6 so as to arrange the imaging circuit 40 at a position corresponding to the parallel mode (step S302). At this time, the projection direction coincides with the initial planning direction. In response to pressing of the fluoroscopy switch by the user, the imaging circuit 40 executes X-ray fluoroscopy of the object P under the control of the imaging control circuit 17 (step S303). An image generation circuit 15 generates data of a fluoroscopic image corresponding to the parallel mode. The display circuit 12 displays, as a moving image, the fluoroscopic image corresponding to the initial planning direction (step S304). The user starts puncture work while confirming the fluoroscopic image displayed on the display circuit 12.

During the puncture work, when the user wants to confirm the length of the puncture needle inserted into the object P, he presses the tilt switch (YES in step S305). Then, the projection mode is switched from the parallel mode to the tilt mode. More specifically, the imaging control circuit 17 controls the C-arm driving mechanism 8 to rotate the C-arm 6 by 5° so as to move the imaging circuit 40 to a position corresponding to the tilt mode (step S306). In the following description, the imaging control circuit 17 controls the imaging circuit 40 in order to interrupt X-ray fluoroscopy while the C-arm 6 is moved. In response to the completion of moving the C-arm 6, the imaging control circuit 17 controls the imaging circuit 40 to restart X-ray fluoroscopy. Even while the C-arm 6 is moved, X-ray fluoroscopy may be performed. The image generation circuit 15 generates data of a fluoroscopic image corresponding to the tilt mode. The display circuit 12 displays the fluoroscopic image corresponding to the tilt mode (step S307). The assistant image generation circuit 21 generates an assistant image based on the fluoroscopic image corresponding to the tilt mode. The display circuit 12 displays the assistant image together with the fluoroscopic image (step S308). The processes in steps S307 and S308 are repetitively executed until the user presses the parallel switch, and the fluoroscopic image corresponding to the tilt mode is displayed on the display circuit 12 (NO in step S309). If the parallel switch is pressed (YES in step S309), the imaging control circuit 17 controls the C-arm driving mechanism 8 to rotate the C-arm 6 by −5° so as to return the imaging circuit 40 to a position corresponding to the parallel mode (step S310). Then, the imaging circuit 40 is arranged at the position corresponding to the parallel mode. The processes in steps S304 to S310 are repetitively executed in accordance with the operation of the tilt switch by the user when, for example, the user confirms whether puncture needle is inserted straight from the insertion target position toward the arrival target position. After confirming that the tip of the puncture needle has come close to the arrival target position, the user presses the lateral switch (YES in step S311). In response to this, the projection mode is switched from the parallel mode to the lateral mode. More specifically, the imaging control circuit 17 controls the C-arm driving mechanism 8 to rotate the C-arm 6 by 90° so as to move the imaging circuit 40 to a position corresponding to the lateral mode (step S312). The image generation circuit 15 generates data of a fluoroscopic image corresponding to the lateral mode. The display circuit 12 displays the fluoroscopic image corresponding to the lateral mode (step S313). The fluoroscopic image corresponding to the tilt mode is displayed on the display circuit 12 until the user presses the parallel switch (NO in step S314). If the parallel switch is pressed (YES in step S314), the process shifts to step S310 to return the imaging circuit 40 to a position corresponding to the parallel mode. After step S310, the process shifts to step S304. The processes in steps S304 to S314 are repetitively executed until the puncture work by the user while performing X-ray fluoroscopy is ended (NO in step S315). When the puncture work is ended and the fluoroscopy switch is released, the first example of the series of workflow operations using the X-ray diagnostic apparatus 1 according to the second embodiment is ended (YES in step S315).

Note that the workflow shown in FIG. 20 describes only two patterns of switching of the projection mode between the parallel mode and the tilt mode, and between the parallel mode and the lateral mode. However, the X-ray diagnostic apparatus 1 can appropriately change the projection mode in accordance with a switch operation by the user. That is, switching of the projection mode may be performed between the tilt mode and the lateral mode. In this case, for example, the process shifts from step S308 to step S311.

The workflow shown in FIG. 20 describes an example in which the projection mode is switched in accordance with a switch operation by the user. However, switching of the projection mode may be automatically performed in accordance with the estimated insertion length.

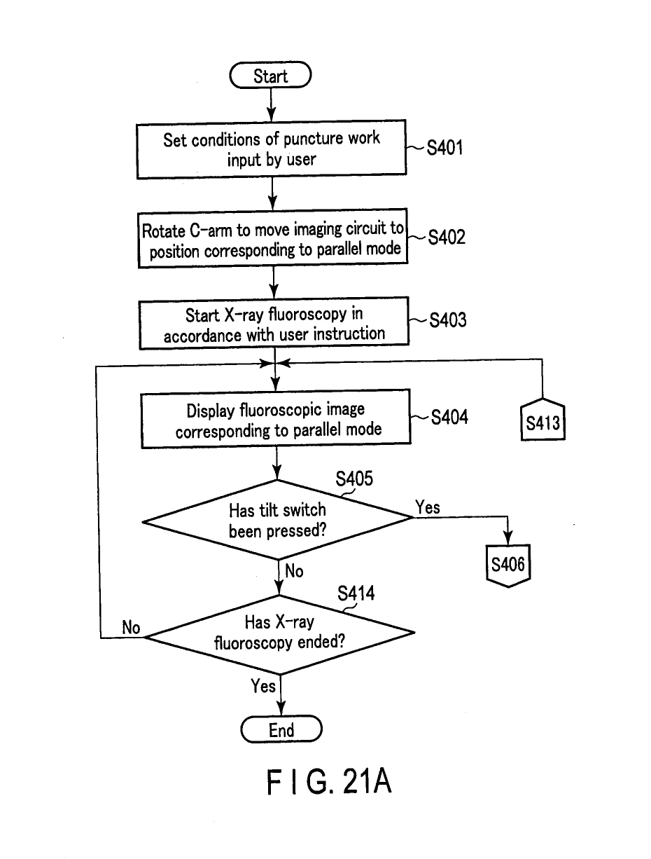

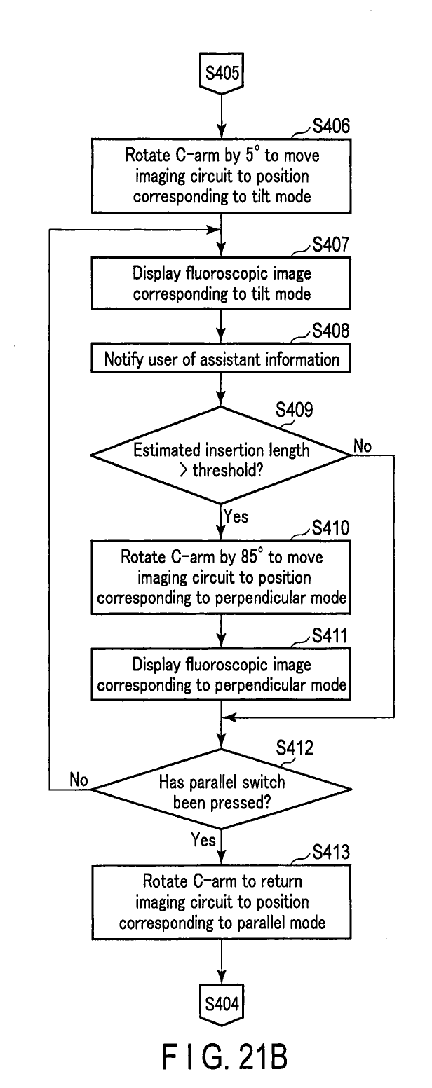

FIG. 21A is a flowchart showing part of the workflow of the second example using the X-ray diagnostic apparatus 1 according to the second embodiment.

FIG. 21B is a flowchart showing a remaining part of the workflow of the second example using the X-ray diagnostic apparatus 1 according to the second embodiment.

In the second example, switching of the projection mode is automatically performed in part. More specifically, when the estimated insertion length is equal to or larger than a threshold after the projection mode is switched from the parallel mode to the tilt mode, the projection mode is automatically switched from the tilt mode to the lateral mode. This threshold corresponds to the ratio of the estimated insertion length to the target insertion length. For example, as the threshold, the ratio of the estimated insertion length to the target insertion length is set to be a value of 90%. A workflow describing procedures is as follows.

Processes in steps S401 to S404 are the same as those in steps S301 to S301 described with reference to FIG. 20.

When the user wants to confirm the length of the puncture needle inserted into the object P, he presses the tilt switch (YES in step S405). Then, the projection mode is switched from the parallel mode to the tilt mode. More specifically, the imaging control circuit 17 controls the C-arm driving mechanism 8 to rotate the C-arm 6 by 5° so as to move the imaging circuit 40 to a position corresponding to the tilt mode (step S406). The image generation circuit 15 generates data of a fluoroscopic image corresponding to the tilt mode. The display circuit 12 displays the fluoroscopic image corresponding to the tilt mode (step S407). The notification circuit 20 notifies assistant information. More specifically, the assistant image generation circuit 21 generates an assistant image based on the fluoroscopic image corresponding to the tilt mode. The display circuit 12 displays the assistant image together with the fluoroscopic image (step S408). The notification circuit 20 compares an estimated insertion length with a threshold. If the estimated insertion length is equal to or larger than the threshold (YES in step S409), the imaging control circuit 17 controls the C-arm driving mechanism 8 to switch the projection mode from the tilt mode to the lateral mode. More specifically, the imaging control circuit 17 controls the C-arm driving mechanism 8 to rotate the C-arm 6 by 85° (i.e., a total of 90° from the parallel mode (initial planning direction)) so as to move the imaging circuit 40 to a position corresponding to the lateral mode (step S410). When the tilt angle θ is “7°”, the C-arm 6 is rotated by 83° by the processing in step S410. The image generation circuit 15 generates data of a fluoroscopic image corresponding to the lateral mode. The display circuit 12 displays the fluoroscopic image corresponding to the lateral mode (step S411). At this time, the display circuit 12 may display the assistant image together with the fluoroscopic image corresponding to the lateral mode. X-ray fluoroscopy in the tilt mode or the lateral mode is performed until the user presses the parallel switch (NO in step S412). If the parallel switch is pressed (YES in step S412), the imaging control circuit 17 controls the C-arm driving mechanism 8 to rotate the C-arm 6 by −90° so as to return the imaging circuit 40 to a position corresponding to the parallel mode (step S413). The imaging circuit 40 is then returned to the position corresponding to the parallel mode. After step S413, the process shifts to step S404. The processes in steps S404 and S413 are repetitively executed until the puncture work by the user while performing X-ray fluoroscopy is ended (NO in step S414). When the puncture work is ended and the fluoroscopy switch is released, the second example of the series of workflow operations using the X-ray diagnostic apparatus 1 according to the second embodiment is ended (YES in step S414).

The workflow using the X-ray diagnostic apparatus 1 according to each of the first and second embodiments described above has the following effects.

The user can selectively use the tilt mode and the lateral mode in accordance with the degree of progress of the puncture needle insertion operation. More specifically, the user performs puncture needle insertion work in the parallel mode. In the tilt mode, the user simply confirms the estimated insertion length and insertion position of the puncture needle on the assistant image. In the lateral mode, the user can confirm the actual insertion length and actual insertion position of the puncture needle on the fluoroscopic image. At the initial stage in which insertion of the puncture needle proceeds, the user simply confirms, on the assistant image, the current degree of progress of the puncture needle and whether the puncture needle is shifted. When the puncture needle comes close to the arrival target position, the user confirms the actual insertion length and insertion position of the puncture needle on the fluoroscopic image. This can shorten the time taken for the conventional puncture needle insertion operation while maintaining the same accuracy of the puncture work as the conventional one. This is because the projection direction is rotated by only the angle θ of smaller than 90° from the parallel mode. The user can easily perform selective use of the projection mode by a switch operation. When the estimated insertion length is equal to or larger than the threshold in the tilt mode, the X-ray diagnostic apparatus 1 can automatically switch the projection mode from the tilt mode to the lateral mode. The number of times of the switch operation by the user can be decreased. Therefore, the X-ray diagnostic apparatus 1 according to each of the first and second embodiments can assist insertion of the puncture needle into the object P by the user.

Processing explained in each of the above-described embodiments may be executed by an apparatus outside the X-ray diagnostic apparatus, for example, a workstation connected to the X-ray diagnostic apparatus.

The functions serving as the respective circuits shown in FIGS. 6 and 17 may be implemented by hardware components such as individual processors or circuitry.

The processing procedures shown in FIGS. 9, 18, 20, and 21 may be executed by properly changing the order.

Some embodiments of the present invention have been described above. However, these embodiments are presented merely as examples and are not intended to restrict the scope of the invention. These novel embodiments can be carried out in various other forms, and various omissions, replacements, and alterations can be made without departing from the spirit of the invention. The embodiments and their modifications are also incorporated in the scope and the spirit of the invention as well as in the invention described in the claims and their equivalents.

1. An X-ray diagnostic apparatus by comprising:
an imaging system configured to rotatably hold, by an arm, an X-ray tube which generates an X-ray, and an X-ray detector which detects an X-ray having passed through an object placed on a top, and generate data of an X-ray image;
a display configured to display the X-ray image generated by the imaging system;
an input circuit configured to input an insertion target position and arrival target position of a puncture needle which is inserted into the object;
an imaging control circuit configured to control the imaging system in order to generate an X-ray image in which an imaging center line connecting a focus of the X-ray tube and a center position of an X-ray detection surface of the X-ray detector has a tilt angle of smaller than 90° with respect to a guideline connecting the insertion target position and the arrival target position;
a puncture needle extraction circuit configured to extract, from the X-ray image generated by the imaging system, a region of the puncture needle inserted in the object;
an insertion length calculation circuit configured to specify an apparent length of the puncture needle based on the region of the puncture needle extracted from the X-ray image having the tilt angle of smaller than 90°, and calculate an estimated insertion length of the puncture needle inserted in the object based on the apparent length of the puncture needle and the tilt angle; and
a notification circuit configured to notify a user of assistant information for assisting an insertion operation of the puncture needle based on the estimated insertion length.

2. The X-ray diagnostic apparatus of claim 1, wherein in order to notify the user of the assistant information, the notification circuit generates an assistant image which corresponds to a direction perpendicular to the guideline and is obtained by superposing a model image of the puncture needle corresponding to the estimated insertion length on an image concerning the object, and outputs the assistant image to the display.

3. The X-ray diagnostic apparatus of claim 2, wherein the notification circuit arranges a first marker indicating the insertion target position at the insertion target position of the puncture needle in the assistant image, and arranges a second marker indicating the arrival target position at the arrival target position of the puncture needle.

4. The X-ray diagnostic apparatus of claim 2, further comprising a storage circuit configured to store volume data concerning the object,
wherein the image concerning the object is an image obtained by executing projection processing on the volume data.

5. The X-ray diagnostic apparatus of claim 2, further comprising a storage circuit configured to store data of a plurality of model images corresponding to respective regions,
wherein the image concerning the object is a model image corresponding to a region of an insertion target of the puncture needle.

6. The X-ray diagnostic apparatus of claim 1, further comprising an input circuit including a first switch configured to switch between a parallel mode in which the imaging center line becomes parallel to the guideline, and a tilt model in which the imaging center line is tilted by smaller than 90° with respect to the guideline,
wherein the imaging control circuit decides a mode of the imaging center line in accordance with an operation of the first switch by the user,
in the parallel mode, the imaging control circuit controls the imaging system to generate by the imaging system an X-ray image in which the imaging center line is parallel to the guideline, and
in the tilt mode, the imaging control circuit controls the imaging system to generate by the imaging system an X-ray image having the tilt angle of smaller than 90°.

7. The X-ray diagnostic apparatus of claim 6, wherein when a ratio of the estimated insertion length to a target insertion length defined by the insertion target position and the arrival target position is higher than a threshold, the imaging control circuit controls the imaging system to generate by the imaging system an X-ray image having a tilt angle of 90° with respect to the guideline, in order to switch to a lateral mode in which the imaging center line becomes perpendicular to the guideline.

8. The X-ray diagnostic apparatus of claim 6, wherein the input circuit includes a second switch configured to switch between the parallel mode and a lateral mode in which the imaging center line becomes perpendicular to the guideline,
the imaging control circuit decides a mode of the imaging center line in accordance with an operation of the first switch and the second switch by the user, and
in the lateral mode, the imaging control circuit controls the imaging system to generate by the imaging system an X-ray image having a tilt angle of 90° with respect to the guideline.

9. The X-ray diagnostic apparatus of claim 6, wherein when switching the mode of the imaging center line from the parallel mode to another mode, the imaging control circuit controls the imaging system to tilt the imaging center line in a direction perpendicular to a long axis of the region of the puncture needle extracted from the X-ray image corresponding to the parallel mode.

10. The X-ray diagnostic apparatus of claim 9, wherein the imaging control circuit controls the imaging system to tilt the imaging center line in a direction in which an angle formed by the imaging center line after switching the mode of the imaging center line from the parallel mode to another mode, and an axis perpendicular to a top surface of the top is decreased, out of a plurality of directions perpendicular to the long axis of the region of the puncture needle.

11. The X-ray diagnostic apparatus of claim 3, wherein the notification circuit arranges a third marker indicating an arrival prospective position, at the arrival prospective position estimated based on an angle formed by the guideline and a long axis of the region of the puncture needle in the assistant image.

12. The X-ray diagnostic apparatus of claim 11, wherein when a gap between the arrival prospective position and the arrival target position is larger than a threshold, the notification circuit notifies the user of a message indicative of a warning.

13. The X-ray diagnostic apparatus of claim 1, wherein the notification circuit generates a sound or voice corresponding to a ratio of the estimated insertion length to a target insertion length defined by the insertion target position and the arrival target position, in order to notify the user of the assistant information.

14. A puncture needle insertion assistant method comprising:
inputting an insertion target position and arrival target position of a puncture needle which is inserted into an object;
extracting a region of the puncture needle inserted in the object, from an X-ray image in which an imaging center line connecting a focus of an X-ray tube and a center position of an X-ray detection surface of an X-ray detector has a tilt angle of smaller than 90° with respect to a guideline connecting the insertion target position and the arrival target position;
specifying an apparent length of the puncture needle based on the region of the puncture needle;
calculating an estimated insertion length of the puncture needle inserted in the object based on the apparent length of the puncture needle and the tilt angle; and
notifying a user of assistant information for assisting an insertion operation of the puncture needle based on the estimated insertion length.
